(19) United States
(12) Patent Application Publication
(10) Pub. No.: US 2020/0275856 A1
(43) Pub. Date: Sep. 3, 2020

(54) AN INTRA- AND CIRCUM-AURAL EEG BRAIN COMPUTER INTERFACE

(71) Applicant: ECOLE DE TECHNOLOGIE SUPERIEURE, Montreal, Quebec (CA)

(72) Inventors: Olivier Valentin, Montreal, Quebec (CA); Guilhem Viallet, Montreal, Quebec (CA); Mikael Ducharme, Montreal, Quebec (CA); Aidin Delnavaz, Lachine, Quebec (CA); Hami Monsarrat-Chanon, Montreal, Quebec (CA); Jeremie Voix, Montreal, Quebec (CA)

(21) Appl. No.: 16/647,818

(22) PCT Filed: Sep. 17, 2018
(86) PCT No.: PCT/CA2018/051153

§ 371 Date: Mar. 16, 2020

Related U.S. Application Data
(60) Provisional application No. 62/559,133, filed on Sep. 15, 2017.

(51) Int. Cl. A61B 5/0478; A61B 5/00

(52) U.S. Cl. CPC A61B 5/0478; A61B 5/6803; A61B 5/7225; A61B 5/6843; A61B 5/6885; A61B 5/6817

(57) ABSTRACT

An electroencephalography (EEG) based brain-computer interface for an ear of a user, the interface having a behind-the-ear piece with a flexible base. The flexible base is shaped to fit mostly behind the ear of a user and has at least one electrode positioned 5 to contact a skin covering a portion of a temporal bone of the user's skull. The flexible base also has a wedge that is shaped to contact an antihelical fold and/or concha of the ear in order to produce and maintain an adequate pressure and contact of the at least one of the plurality of electrodes on a portion of skin covering a temporal bone of the user's skull. The interface is adapted to produce voltage fluctuations measured by 0 the electrodes for determining a brain electrical activity. A system for determining a brain activity indicator using the electroencephalography (EEG) based brain-computer interface.

### CROSS-REFERENCE TO RELATED APPLICATIONS

The present patent application claims the benefits of priority of U.S. Provisional Patent Application No. 62/559,133, entitled “An Intra- and Circum-aural EEG Brain Computer Interface” and filed at the United States Patent and Trademark Office on Sep. 15, 2017, the content of which is incorporated herein by reference.

### TECHNICAL FIELD

The present relates to brain activity recording by electroencephalography. More particularly the present relates to a brain-computer interface for brain activity recording by electroencephalography.

### BACKGROUND

Brain-computer interfaces (BCI) can directly translate human intentions into discrete commands while bypassing the human locomotor system. Most non-invasive BCI systems currently in use are based on electroencephalography (EEG) recording technology using recent developments of mobile EEG solutions. However, current non-invasive BCI systems still have important limitations. Although the current systems may be robust to motions and can make abstraction of human body movements, the current systems can be cumbersome and visible to others which can be inadequate for being used in social settings or during physical activity. Indeed, sensors of mobile EEG-based BCI systems are not inconspicuous enough for use in social settings and can be cumbersome while performing a physical activity such a running, swimming, cycling, etc.

For instance, when trying to measure Auditory Steady State Responses (ASSRs) which are recordable electrophysiological responses, at least one electrode 120 such as presented in Prior Art FIG. 1B is strategically placed on the human scalp to capture brain activity. ASSRs correspond to the brain activity evoked by one or more stimuli which are characterized by carrier frequencies (Fc) that are amplitude-modulated at a specific frequency (Fm). In practice, when a person is exposed to such stimuli, spectral power of the EEG frequency spectrum that is related to the stimuli will manifest at Fm, and may also appear at its harmonics. ASSRs' recordings and stimuli generations were conducted by using the Multiple Auditory Steady-State Evoked Response, known as the “MASTER System™”, a LabVIEW™ based software developed at the Rotman Research Institute, an international center for the study of human brain function.

The MASTER System™ is a data acquisition system designed by Michael S. John and Terrence W. Picton to assess human hearing by recording auditory steady-state responses. The LabVIEW™ based environment simultaneously generates multiple amplitude-modulated and/or frequency-modulated auditory stimuli, acquires electrophysiological responses to these stimuli, displays these responses in the frequency-domain, and determines whether or not the responses are significantly larger than background physiological activity.

Prior Art FIG. 1A presents various hardware components of the MASTER system 100. The MASTER System™ 100 includes a PC 101, an acquisition board 104, a variable gain amplifier 106, an audiometer 108, a transducer 110 (usually, earphones or headphones), an EEG amplifier 112, coaxial cables and audio cables. In addition, as concurrently presented in Prior Art FIG. 1B, the system 100 uses a set of gold-plated electrodes 120, such as a captor electrode 122 placed at vertex (Cz), a reference electrode 124 placed on the back of the neck (near hairline) and a ground electrode 126 placed on the clavicle.

All components of the MASTER System™ 100 are monitored by the single PC 101. The stimulation signals from the analogue output of the NI-USB 6229 board 104 are attenuated by an operational amplifier 106 with a gain of −0.5, so that they may be delivered to the “CD input” of the audiometer 108, which enables the operator to adjust the levels of stimuli delivered by a transducer (such as earphones or headphones). In parallel, ASSRs are scalp-recorded by the electrodes (122,124 and 126) placed at vertex (+) 122, hairline (ref) 124 and clavicle (ground) 126 and are then amplified by the EEG amplifier 112, before reaching the analogue input of the data acquisition board 104 connected to the computer 101. Data is processed online with the LabVIEW™ based software.

Prior art FIG. 1C presents an EEG capture device 140 having circum-aural electrodes positioned on a semi-flexible plastic base material. A conductive paste must be applied to the skin to provide proper electrode contact and an adhesive film (not shown) is applied over the device 140 to secure the device 140 in a desired position and further ensure a proper skin-electrode contact. The electrodes are positioned such as to contact a mastoid area as well as a lower jaw bone area. Movement of the lower jaw bone can compromise the proper positioning of the device 140 and prevent effective brain signal acquisition. Moreover, the shape and size of the device 140 make it visible to others which can be inadequate for being used in social settings.

Others have developed portable EEG monitoring systems. For instance, Kidmose et al. describe in US Patent publications 2012/0123290 and 2012/0302858 an EEG monitoring system adapted to be carried continuously by a person to be monitored. The system has an implant unit that is located subcutaneously behind the ear of a patient. The implant unit has an electronics part and two electrodes for picking up electrical EEG signals from the brain of the patient. The electronics part has the necessary electronics for sampling the EEG signals measured by the electrodes and transmitting them wirelessly to an external monitoring unit. The monitoring unit resembles a behind-the-ear hearing aid having an earplug and a housing that is placed behind the ear. The housing has a processing unit adapted to receive wirelessly the EEG readings from the implant unit. The housing is connected to the earplug via a sound tube or an electric cord leading to a receiver of the earplug. This allows the monitoring unit to transmit messages, such as alarms or warnings, into the ear of the person carrying the EEG monitoring system. Despite the portability of the system, this system requires surgery to position the electrodes and the electronics part subcutaneously behind the ear of the patient and is invasive. Moreover, the patient cannot easily remove the implant unit at his own will.

In U.S. Pat. No. 9,408,552 to Kidmose et al., there is described an earplug having a shell with at least two electrodes adapted to measure brain wave signals. The electrodes are positioned on a contour portion of the shell and are connected to a processor for measuring the signals. The shell is shaped to individually match at least part of the ear canal and the concha of the user. The earplug is connected to a behind-the-ear component and the brain wave signals detected by the electrodes of the earplug are transmitted to the behind-the-ear component for further processing. The shell is made from a flexible material such as plastic or silicon. The electrodes are positioned on or integrated within the surface of the shell and counts at least one reference electrode and at least one detecting electrode. Kidmose et al. present an earplug having more or less five electrodes. The electrodes are made from alloys such as stainless steel, platinum-iridium or noble metals such as silver, titanium, platinum and tungsten. Otherwise, the electrodes can also be made from silver-silver chloride. In order to improve the quality of the signals detected by the electrodes, a conductive gel is applied. Although Kidmose et al. describe a portable and non-invasive brain wave signal measuring device, since the active electrode or captor electrode is positioned in proximity with the reference electrode, the electrodes can only measure localised brain activity produced by cortex generators that are in proximity with the outer ear-canal and may not be appropriate for providing general or extensive brain activity readings.

### SUMMARY

According to one aspect, there is an electroencephalography (EEG) based brain-computer interface for an ear of a user. The interface has a behind-the-ear piece. The behind-the-ear piece has a flexible base that is shaped to fit mostly behind the ear of a user. The flexible base has at least one electrode positioned to contact with a portion of skin covering a temporal bone of the user's skull when the device is worn and the at least one electrode is selected from a group consisting of a reference electrode, at least one captor electrode and a ground electrode. The reference electrode is configured to measure a first voltage fluctuation. The at least one captor electrode is configured to measure a second voltage fluctuation. The ground electrode is configured to measure a third voltage fluctuation. The flexible base also has a wedge portion that is shaped to contact at least in part an antihelical fold and/or concha of the ear in order to produce and maintain an adequate pressure and contact of the at least one electrode on a portion of skin covering a temporal bone of the user's skull. The interface is configured to provide the first voltage fluctuation, the second voltage fluctuation and the third voltage fluctuation for determining a brain electrical activity.

According to another aspect, there is an electroencephalography (EEG) based brain-computer interface for an ear of a user. The interface has two in-ear pieces. The first in-ear piece has an ear canal engaging member having a reference electrode configured to measure a first voltage fluctuation and being shaped to engage an outer-ear canal of a first ear in order to allow the reference electrode to contact at least in part a wall of an outer ear canal. The second in-ear piece has an ear canal engaging member having at least one captor electrode configured to measure a second voltage fluctuation and being shaped to engage an outer-ear canal of a second ear to allow the at least one captor electrode to contact at least in part a wall of an outer ear canal. One of the first in-ear piece and the second in-ear piece further has a ground electrode configure to measure a third voltage fluctuation. The interface is configured to provide at least one of the first voltage fluctuation, the second voltage fluctuation and third voltage fluctuation for determining a brain electrical activity.

According to yet another aspect, there is a system for determining a brain activity indicator using a brain-computer interface. The system has an electroencephalography (EEG) based brain-computer interface as defined above, a differential amplifier and a computer device. The amplifier is configured to amplify and convert into a digital form the first voltage fluctuation, the second voltage fluctuation and the third voltage fluctuation provided by the brain-computer interface and to produce associated amplified and converted voltage fluctuations. The computer device is configured to determine a brain electrical activity indicator according to the associated amplified and converted voltage fluctuations.

The features of the present invention which are believed to be novel are set forth with particularity in the appended claims.

### BRIEF DESCRIPTION OF THE DRAWINGS

Further features and advantages of the present invention will become apparent from the following detailed description, taken in combination with the appended drawings, in which:

FIG. 1A presents a prior art Auditory Steady State Responses (ASSR) recording system;

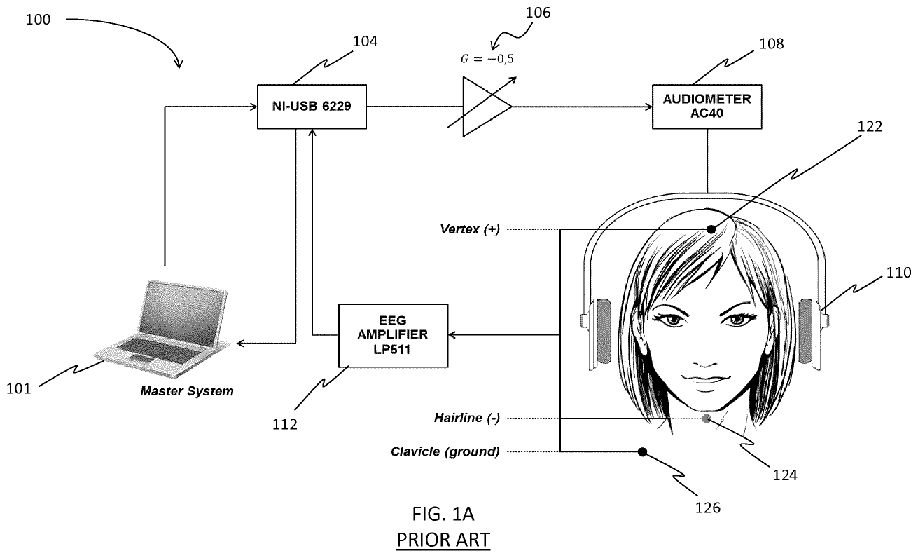

FIG. 1B presents a prior art gold foil and a prior art gold-plated cup electrodes;

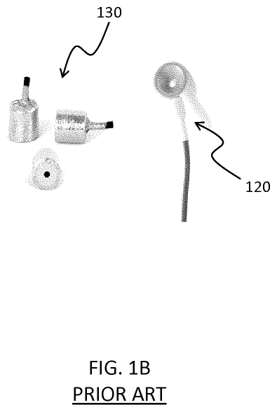

FIG. 1C presents a prior art EEG capture device having circum-aural electrodes positioned on a semi-flexible plastic base material;

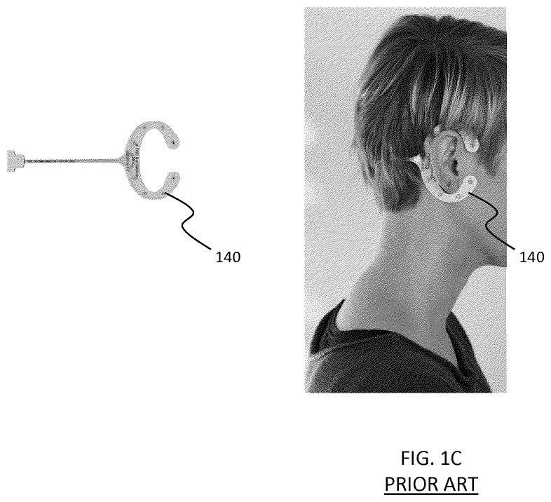

FIG. 2A presents an ear device having an in-ear piece and a behind-the-ear piece, according to one embodiment;

FIG. 2B presents an ear device having a behind-the-ear piece, according to an alternate embodiment;

FIG. 2C presents the ear device of FIG. 2A or FIG. 2B being connected to an ASSR acquisition system, according to one embodiment;

FIG. 2D presents the ear device of FIG. 2A or FIG. 2B being connected to a differential amplifier for further processing by a computerized system configured to determine a brain electrical activity, according to one embodiment;

FIG. 2E presents an ear device having a two in-ear pieces, according to one embodiment;

FIG. 2F presents alternate in-ear pieces of the in-ear pieces of FIGS. 2A and 2E, according to one embodiment;

FIG. 3A presents a drawing of a posterior auricle portion of an ear and the various bones of the human skull, in order to describe the positioning of the ear device, according to one embodiment;

FIG. 3B presents two photographic drawings of a person wearing the behind-the-ear pieces of FIG. 2A and of FIG. 2B, according to one embodiment;

FIG. 4A presents a table describing electrodes' placements used for two experiments, «G.P.C» refers to gold-plated cup electrodes, «G.F» refers to gold-foil electrodes and «EAR» refers to ear device electrodes;

FIG. 4B presents a chart depicting Signal-to-noise ratio, in dB, of ASSRs scalp-recorded on subject #1 using gold electrodes (control condition #1 and #2) and the electrodes of the behind-the-ear piece, according to one embodiment; and

FIG. 4C presents a chart depicting Signal-to-noise ratio, in dB, of ASSRs scalp-recorded on subject #3 using gold electrodes (control condition #1 and #2) and the electrodes of the in-ear piece, according to one embodiment.

It will be noted that throughout the appended drawings, like features are identified by like reference numerals.

### DETAILED DESCRIPTION

A novel intra- and circum-aural EEG brain computer interface will be described hereinafter. Although the invention is described in terms of specific illustrative embodiment(s), it is to be understood that the embodiment(s) described herein are by way of example only and that the scope of the invention is not intended to be limited thereby.

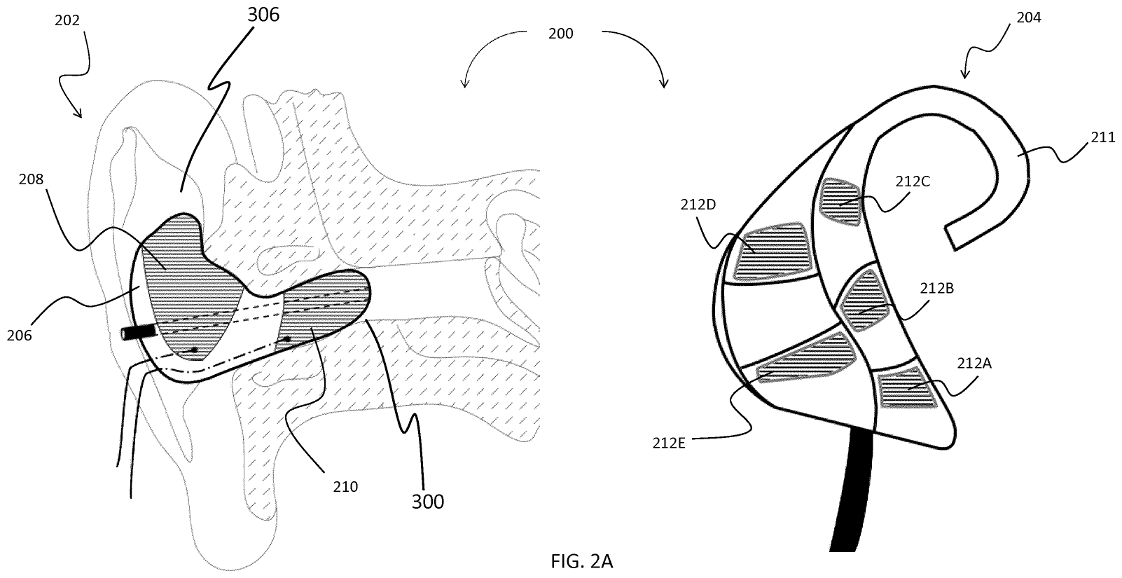

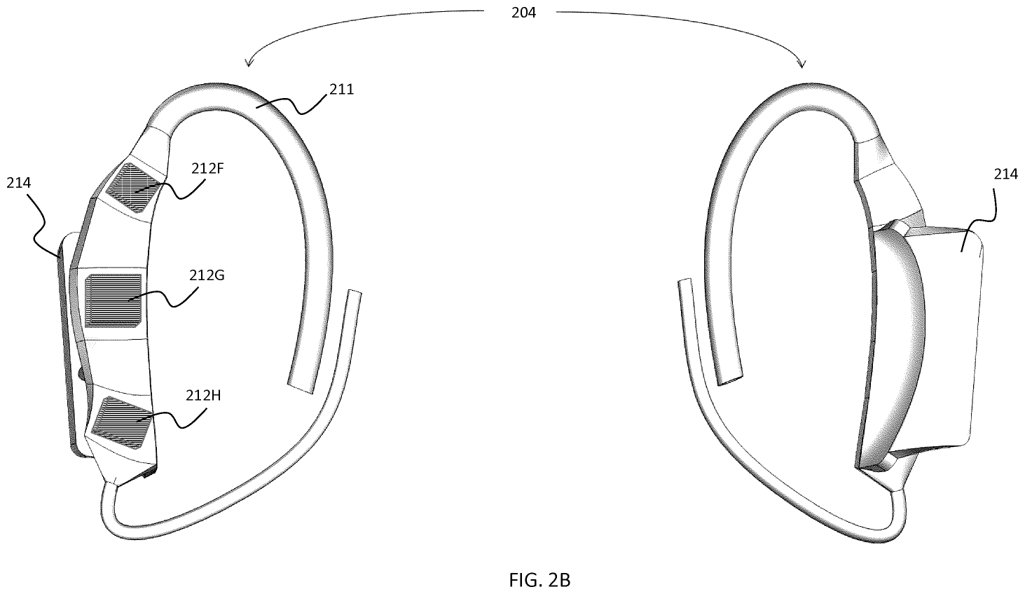

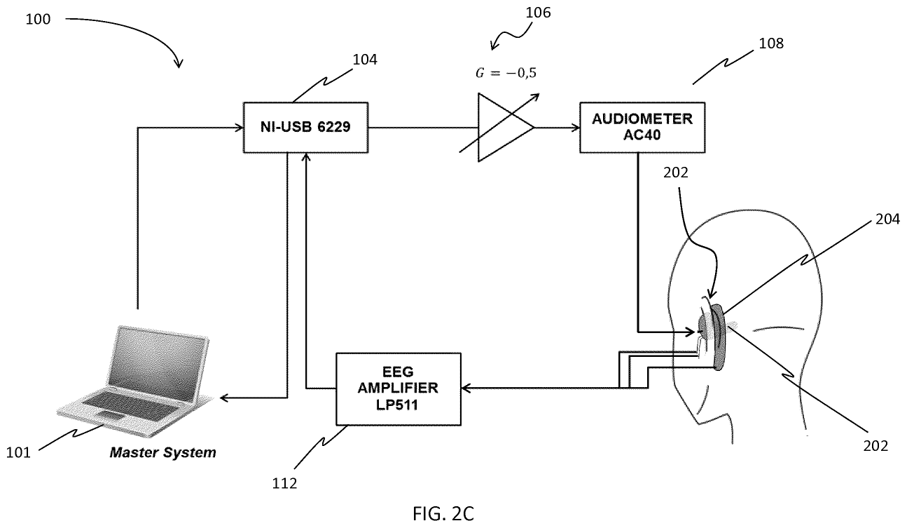

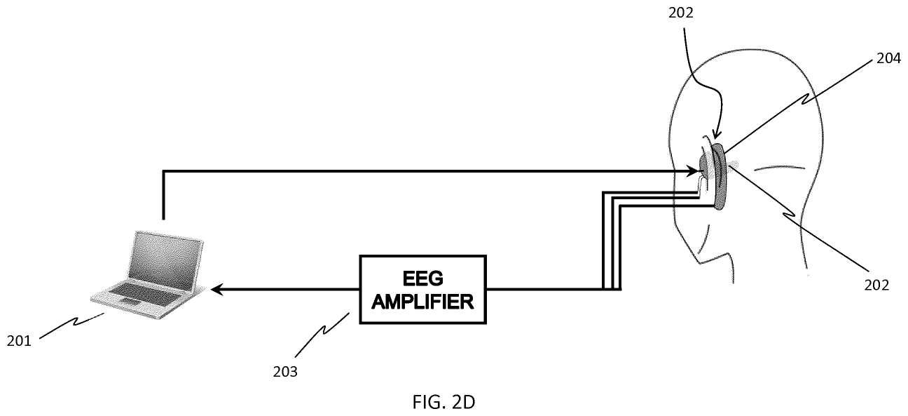

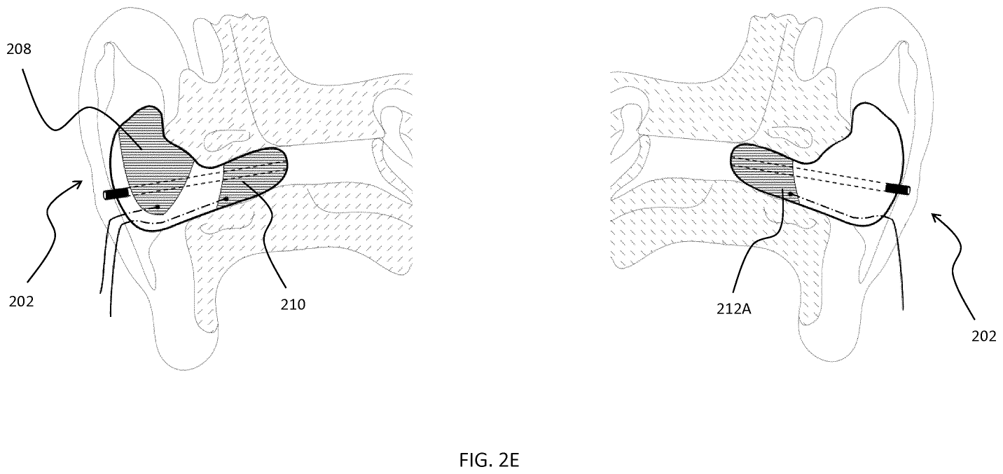

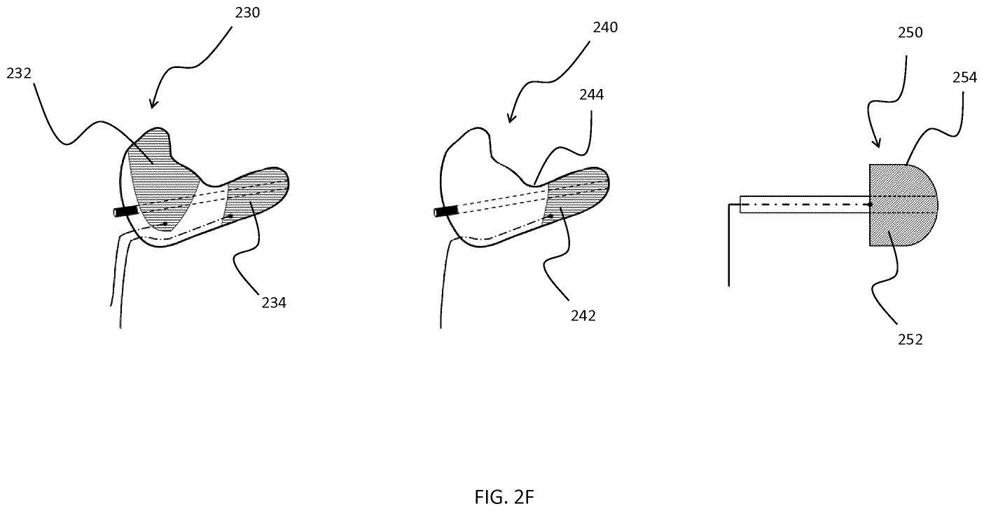

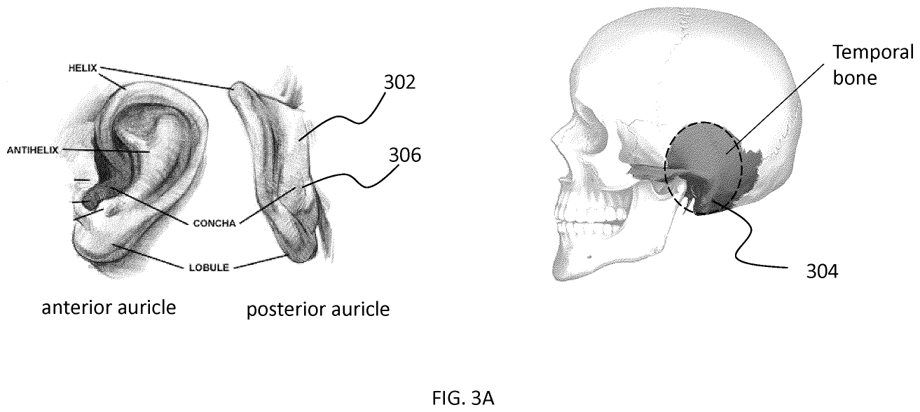

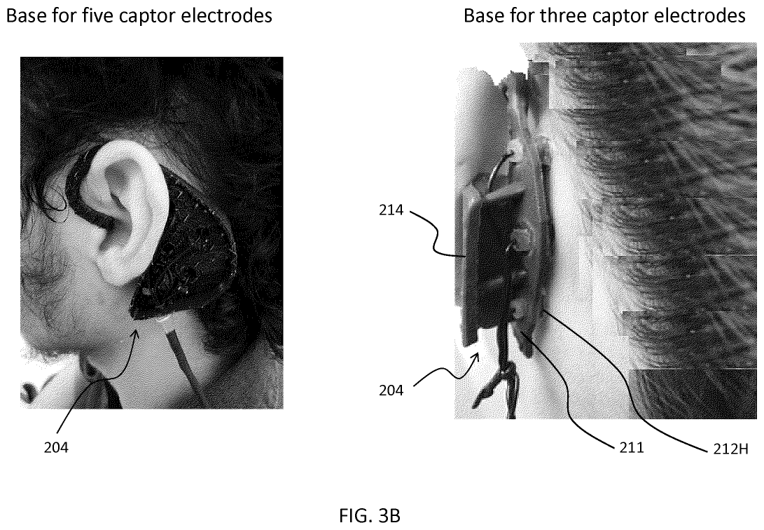

Presented in FIG. 2A is an ear device 200 that has an in-ear piece 202 and a behind-the-ear piece 204, according to one embodiment. The in-ear piece 202 has an ear canal engaging member 206 such as an earplug. The ear canal engaging member 206 is shaped and formed to contact at least in part the walls 300 of an outer ear canal. The ear canal engaging member 206 has an integrated ground electrode 208 and an integrated reference electrode 210. The integrated ground electrode 208 and integrated reference electrode 210 are strategically positioned within the engaging member 206 such that an adequate pressure from the walls of the outer ear-canal and the concha 306 of the ear provide an effective contact, in order to obtain an adequate impedance matching between the skin and the in-ear electrodes (208 and 210), as concurrently presented in FIG. 3A.

The behind-the-ear piece 204 is adapted to contact the skin covering the skull opposite or near the antihelical fold 302 of the ear, as presented in FIGS. 2A and 3A. The behind-the-ear piece 204 is also adapted to contact a portion of the skin covering the temporal bone of the human skull such as the mastoid area 304 as depicted with dotted lines in FIG. 3A.

As further presented in FIG. 2A, the behind-the-ear piece 204 has a flexible base 211 to which are strategically positioned five captor electrodes (212A, 212B, 212C, 212D, 212E). The captor electrodes 212A, 212B and 212C are positioned in order to be placed in contact with the skin covering the skull opposite or in proximity with the antihelical fold 302, when the device 200 worn, as concurrently presented in FIG. 3A. The captor electrodes 212D and 212E are positioned in order to be placed in contact with a portion of the skin covering the temporal bone 304 of the human skull such as the mastoid area, when the device 200 worn. It shall be recognized that the portion of the temporal bone 304 depicted in FIG. 3A can differ in shape and size depending on the positioning of the captor electrodes 212D and 212E on or within the behind-the-ear piece 204 and the ear morphology of the user.

Presented in FIG. 2B, is an alternate embodiment of the behind-the-ear piece 204. In this embodiment, the flexible base 211 has three strategically positioned captor electrodes (212F, 212G, 212H) in order to be placed in contact with the skin covering the skull opposite or in proximity with the antihelical fold 302, when the device 200 worn.

According to one embodiment, the behind-the-ear piece 204 also has a comfort wedge 214 positioned to contact at least in part the antihelical fold 302 and/or the concha 306 in order to produce and maintain an adequate pressure and contact of the captor electrodes on the skin of the skull opposing the antihelical fold 302 and/or concha 306.

In the embodiment of the behind-the-ear piece 204 presented in FIG. 2B, the comfort wedge 214 is integral with the flexible base 211. However, it shall be recognized that in another embodiment, that the comfort wedge 214 may be removeable. Moreover, the comfort wedge 214 can be replaced by any other suitable comfort wedge having a size and shape that corresponds to the space defined between the antihelical fold 302 and/or concha 306 and the opposing skull region of a user. According to yet another embodiment, the flexible base 211 has an integral first comfort wedge having a minimal width and a second comfort wedge that is removably attachable to the first comfort wedge in order to suitably increase a width of the resulting comfort wedge, depending on the ear morphology.

In the embodiment of the behind-the-ear piece 204 presented in FIG. 2B, the comfort wedge 214 is flexible. It shall however be recognized that degree of flexibility of the comfort wedge 214 can differ from one embodiment to another and that in some cases, the comfort wedge 214 can be made from a harder material, without departing from the scope of the present solution.

Moreover, in the embodiment presented in FIG. 2B, the base 211 is flexible. It shall however be recognized that degree of flexibility of the base 211 can differ from one embodiment to another and that in some cases, the base 211 can be made from a harder material, without departing from the scope of the present solution.

According to one embodiment, the captor electrodes (212A, 212B, 212C, 212D, 212E, 212F, 212G and/or 212H) are made of a soft biocompatible polymer material, such as medical grade silicon, filled with a conductive material, such as carbon chopper. The silicon filled with carbon chopper has an adequate conductivity while remaining resilient in order to adapt with comfort to the shape of the posterior auricle of the wearer. According to one embodiment the carbon chopper is mixed with silicon at a weight ratio ranging from 0.5% to 3%. According to another embodiment the carbon chopper is mixed with silicon at a weight ratio ranging from 0.5% to 2%. According to yet another embodiment the carbon chopper is mixed with silicon at a weight ratio ranging from 0.5% to 1%. According to another embodiment, the carbon chopper is mixed with silicon of around a ratio of 0.6%. For instance, for 43 grams of silicon, 0.25 grams of carbon is added.

According to one embodiment, the captor electrodes (212A, 212B, 212C, 212D, 212E, 212F, 212G and 212H) are positioned on the base 211 as depicted in FIGS. 2A and 2B. However, it shall be recognized that the captor electrodes can be positioned within the base 211 or can also be integral with the base 211.

The shape and size of the ear device 200 is adapted to obtain voltage fluctuation measurements with the electrodes (208, 210, 212A, 212B, 212C, 212D, 212E, 212F, 212G and 212H) while seamlessly being worn in and/or around the ear. Indeed, the device 200 generally aims at not being cumbersome to the user and to be used in social setting without drawing too much attention.

As presented in FIG. 2C or 2D, the ear device 200 is adapted to provide to an analysis system 101 or 201 voltage fluctuation measurements indicative of brain activity. The voltage fluctuation measurements are measured by the ground electrode 208, by the reference electrode 210 and by at least one of the captor electrodes (212A, 212B, 212C, 212D, 212E, 212F, 212G and 212H) according to a variety of cortex generators and are amplified and converted into a digital form by a differential amplifier 112 or 203. Based on the amplified and converted voltage fluctuation measurements the data analysis system 101 or 201 can produce electroencephalography recordings. The analysis system 101 or 201, the differential amplifier 112 or 203 and the ear device 200 are connected via a wire connection or via a wireless connection.

According to one embodiment, the analysis system 101 or 201 is adapted to produce a predetermined stimulus and expose the user to the predetermined stimulus. During the predetermined stimulus, the brain-computer interface is configured to measure the voltage fluctuations. The analysis system 101 or 201 then analyses the voltage fluctuations associated to the produced predetermined stimulus. The predetermined stimulus can be a sound stimulus, a visual stimulus or any other kind of stimulus know to produce brain activity.

It shall be recognized that in some embodiments, the ground electrode and/or the reference electrode can be positioned on the flexible base 211 and that the in-ear piece 202 may not be required.

It shall further be recognized that the data analysis system 201 or 101 can produce electroencephalography recordings based on voltage fluctuation measurements provided by two devices 200 worn by a user on each ear. Indeed, the device 200 can be worn on each ear of the user and the analysis system 201 or 101 may provide EEG results with greater accuracy, particularly when relying on contralateral cross-referencing. Moreover, in one mode of operation, the device 200 having only a ground and a reference electrode is worn on one ear and the device 200 having a suitable number or captor electrodes is worn on the other ear of the user.

For instance, in some embodiments, as presented in FIG. 2E, the ground electrode 208 and the reference electrode 210 are positioned on one in-ear piece 202 that is adapted to be inserted into one of the user's ears. At least one of the captor electrodes (212A, 212B, 212C, 212D, 212E, 212F, 212G and 212H) is positioned on another in-ear piece 202 adapted to be inserted into another one of the user's ears. Notice that the ground electrode 208 can be positioned on either one of the in-ear pieces 202, without departing from the scope of the present device 200.

In other embodiments, the ground electrode and/or the reference electrode is positioned on one behind-the-ear piece 204 and at least one of the captor electrodes (212A, 212B, 212C, 212D, 212E, 212F, 212G and 212H) is positioned on another behind-the-ear piece 204.

The data analysis system 201 or 101 is indeed adapted to provide EEG results based on either ipsilateral (same side) EEG readings provided by a device 200 worn on one ear, as presented in FIG. 2D. The data analysis system 201 is also adapted to provide EEG results based on contralateral (opposite side) EEG readings provided by a device 200 worn on both ears of a user. Indeed, as presented in FIG. 2E, a first in-ear piece 202 has a reference electrode (R) 210 adapted to measure the voltage fluctuations to be used as the reference electrical potential for the computation of the brain electrical activity. A second in-ear piece 202 has at least one captor electrode (C) 212A to measure the voltage fluctuations to be used as the active electrical potential for the computation of the brain electrical activity. Either one of the in-ear pieces 202 comprises a ground electrode (G) 208 adapted to measure electrical noise and to prevent power line noise from interfering with the computation of the brain electrical activity. The computation of the brain electrical activity performed by the analysis system 201 corresponds to the difference between the CG voltage and the RG voltage (i.e. CG minus RG). Notice that for contralateral EEG readings one or both of the in-ear pieces 202 can be replaced by a behind-the-ear piece 204, without departing from the present device 200.

It shall be recognized that the captor electrodes (212A, 212B, 212C, 212D, 212E, 212F, 212G and 212H) may have any suitable shape, placement or orientation and may vary in number from one embodiment to another without departing from the scope of the present invention. For instance, the placement and number of electrodes as shown in the behind-the-ear pieces 204 of FIGS. 2A and 2B produce effective readings among a number of people each having a different yet most common ear morphologies.

It shall further be recognized that the in-ear piece 202, can have a variety of shapes and a variety of number of electrodes. For instance, as presented in FIG. 2F, the in-ear piece 202 can be a customized ear-piece (230 and 240) that is molded according to the outer-ear canal shape of the user. The in-ear piece 202 can also be a generic ear-piece 250 that is shaped or that has a modifiable shape to adequately fit within the outer-ear canal of all users or for a range of users. As further presented in FIG. 2F, the in-ear piece 202 can have a plurality of electrodes (232 and 234) such as the dual electrode ear-piece 230. The in-ear piece 202 can also comprise a single electrode such as the single electrode ear-pieces (240 and 250). Depending on the ear device 200 configuration, the single electrode is either a captor electrode, a ground electrode or a reference electrode. Notice that the single electrode ear-piece 240 has one electrode 242 that is shaped to occupy a partial surface of an ear-canal engaging member 244. However, the single electrode ear-piece 250 has one electrode 252 that is shaped to occupy an entire surface of an ear-canal engaging member 254.

A skilled person shall recognize that if the base 211 were custom molded or printed to properly fit a specific ear morphology the placement and the number of captor electrodes may be reduced to two or three, without departing from the scope of the present ear device 200.

It shall be recognized that the captor electrodes (212A, 212B, 212C, 212D, 212E, 212F and 212G), the ground electrode 208 and the reference electrode 210 can be used as dry or wet electrodes. When used as wet electrodes a conductive paste is be applied to the skin.

According to one embodiment, the shape and size of the base 211 and the shape size of the electrodes (212A, 212B, 212C, 212D, 212E, 212F, 212G and 212H) are defined according to an outer ear impression of a user, in order to obtain a customized fit for the user. According to another embodiment, the shape and size of the bases 211A and 211B and the shape and size of the electrodes (212A, 212B, 212C, 212D, 212E, 212F, 212G and 212H) are defined according to an outer ear impression taken from a plurality of participants in order to obtain an adequate skin contact for a larger group of people. The shape and size of the present behind-the-ear piece 204 presented in FIGS. 2A and 2B were determined according to outer ear impressions taken from ten participants.

According to one embodiment, the shape and size of the canal engaging member 206 and the shape and size of the ground and reference electrodes (208 and 210) are defined according to an ear canal impression of a user, in order to obtain a customized fit for the user. According to another embodiment, the shape and size of the canal engaging member 206 and the shape and size of the ground and reference electrodes (208 and 210) are defined according to an ear canal impression taken from a plurality of participants in order to obtain an adequate skin contact for a larger group of people. The shape and size of the in-ear piece 202 presented in FIG. 2A was determined according to in-ear impressions taken from ten participants.

Additive manufacturing and casting techniques have been used to produce the present behind-the-ear piece 204. It shall however be recognized that other techniques such as etching and molding are also possible to produce the behind-the-ear piece 204.

It shall be recognized that the ear device 200 could be integrated with other audio devices, such as hearing aids and headphones, to build next-generation devices that dynamically adapt to the listener's intentions and cognitive state changes.

Experiment

The present study evaluates the signal quality of auditory steady state responses (ASSRs) obtained with the unobtrusive ear device 200, incorporating in- and around-the-ear electrodes and compared to those obtained with well-established gold-plated electrodes.

In one experiment, five men aged between 19 years and 29 years and having hearing thresholds below 20 dB HL (from 125 Hz to 8 kHz) were assessed.

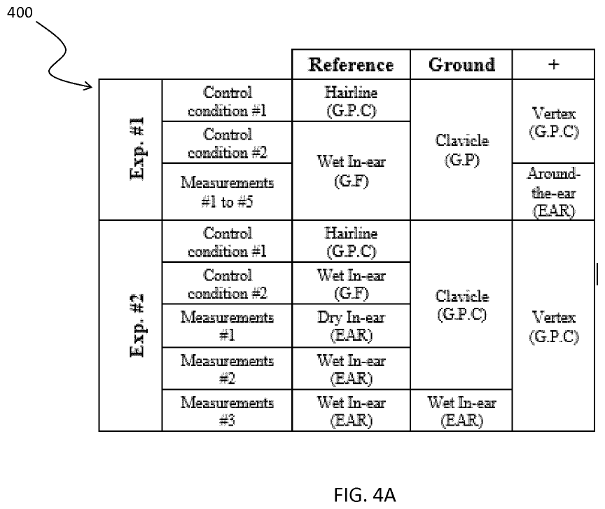

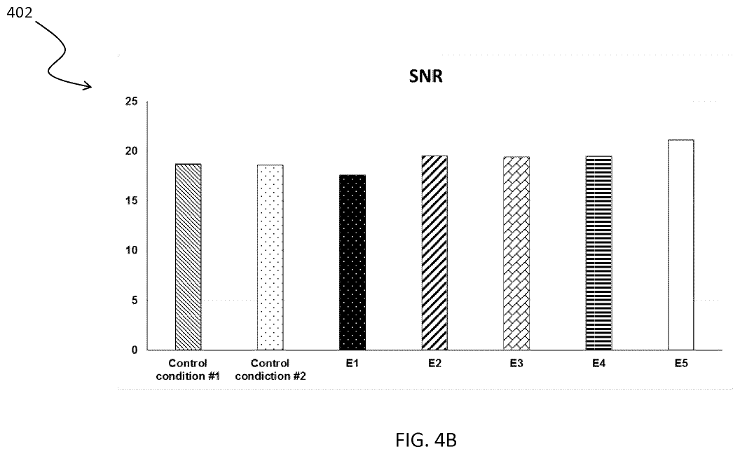

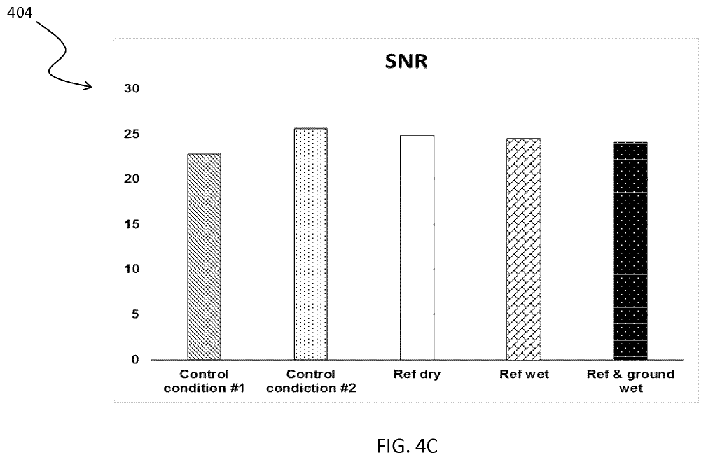

A typical experiment procedure included two recording sessions which purpose was to compare ASSRs scalp-recorded with the behind-the-ear piece 204 and in-ear piece 202 to those obtained with gold foil 130 or gold-plated cup electrodes 120. For both experiments, the stimuli consisted of four pure tones (500, 1000, 2000 and 4000 Hz) amplitude modulated at 40 Hz with a depth of 100%. The different placements used for each experiment are reported in the table 400 of FIG. 4A.

Although the ear device 200 signals show lower amplitudes, corresponding signal-to-noise ratios of ASSRs recorded with the ear device 200 were similar to those of ASSRs recorded with gold electrodes (120 or 130), as presented in graphs 402 and 404 of FIGS. 4B and 4C. As a consequence, the proposed ear device 200 seems to be a promising candidate for future small, mobile, and unobtrusive BCI platforms.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1) An electroencephalography (EEG) based brain-computer interface for an ear of a user, the interface comprising:
a behind-the-ear piece, the behind-the-ear piece comprising a flexible base shaped to fit mostly behind the ear of a user, the flexible base comprising:
at least one of a plurality of electrodes positioned to contact with a portion of skin covering a temporal bone of the user's skull when the device is worn;
the plurality of electrodes comprising a reference electrode configured to measure a first voltage fluctuation, at least one captor electrode configured to measure a second voltage fluctuation and a ground electrode configured to measure a third voltage fluctuation;
a wedge portion that is shaped to contact at least in part an antihelical fold and/or concha of the ear in order to produce and maintain an adequate pressure and contact of the at least one of the plurality of electrodes on a portion of skin covering a temporal bone of the user's skull; and
the interface being configured to provide the first voltage fluctuation, the second voltage fluctuation and the third voltage fluctuation for determining a brain electrical activity.

2) The brain-computer interface of claim 1, wherein the at least one of the plurality of electrodes is positioned to contact a portion of skin covering the temporal bone opposite of an antihelical fold of the ear.

3) The brain-computer interface of claim 1, wherein the at least one of the plurality of electrodes is positioned to contact a portion of skin covering a mastoid portion of the temporal bone.

4) (canceled)

5) (canceled)

6) The brain-computer interface of claim 1, further comprising an in-ear piece, the in-ear piece having an ear canal engaging member, the ear canal engaging member having at least another one of the plurality of electrodes positioned to contact a wall of the outer ear canal.

7) (canceled)

8) The brain-computer interface of claim 6, wherein the ear canal engaging member is shaped such that an adequate pressure from the walls of the outer ear-canal and the concha of the ear provides a contact producing an adequate impedance matching between the skin and the at least one of the plurality of electrodes.

9) The brain-computer interface of claim 8, wherein the at least one of the plurality of electrodes is made of a soft biocompatible polymer material filled with a conductive material.

10) The brain-computer interface of claim 9, wherein the conductive material is carbon chopper.

11) The brain-computer interface of claim 10, wherein the soft biocompatible polymer material is silicon and the silicon is filled with carbon chopper according to a weight ratio ranging from 0.5% to 3%.

12) The brain-computer interface of claim 11, wherein the silicon is filled with carbon chopper according to a weight ratio ranging from 0.5% to 1%.

13) The brain-computer interface of claim 1, where the interface is an audio ear device.

14) The brain-computer interface of claim 1 further comprising a differential amplifier being configured to amplify and to convert into a digital form the first voltage fluctuation, the second voltage fluctuation and the third voltage fluctuation and to produce associated amplified and converted voltage fluctuations adapted to determine a brain electrical activity according to at least the associated amplified and converted voltage fluctuations.

15) The brain-computer interface of claim 14 wherein the interface is configured to transmit the associated amplified and converted voltage fluctuations to an analysis system configured to determine a brain electrical activity according to at least the associated amplified and converted voltage fluctuations.

16) An electroencephalography (EEG) based brain-computer interface for an ear of a user, the interface comprising:
a first in-ear piece comprising a first ear canal engaging member, the first ear canal engaging member comprising a reference electrode configured to measure a first voltage fluctuation and being shaped to engage an outer-ear canal of a first ear in order to allow the reference electrode to contact at least in part a wall of an outer ear canal;
a second in-ear piece comprising a second ear canal engaging member, the second ear canal engaging member comprising at least one captor electrode configured to measure a second voltage fluctuation and being shaped to engage an outer-ear canal of a second ear to allow the at least one captor electrode to contact at least in part a wall of an outer ear canal;
one of the first and second in-ear pieces further comprising a ground electrode configure to measure a third voltage fluctuation; and
the interface being configured to provide at least one of the first voltage fluctuation, the second voltage fluctuation and third voltage fluctuation for determining a brain electrical activity.

17) The brain-computer interface of claim 16 further comprising differential amplifier configured to amplify and to convert into a digital form the first voltage fluctuation, the second voltage fluctuation and the third voltage fluctuation and to produce associated amplified and converted voltage fluctuations for determining a brain electrical activity according to at least the associated amplified and converted voltage fluctuations.

18) (canceled)

19) (canceled)

20) (canceled)

21) (canceled)

22) (canceled)

23) The brain-computer interface of claim 16, the interface is configured to transmit the associated amplified and converted voltage fluctuations to an analysis system configured to determine a brain electrical activity according to at least the associated amplified and converted voltage fluctuations.

24) A system for determining a brain activity indicator using a brain-computer interface, the system comprising:
an electroencephalography (EEG) based brain-computer interface as claimed in any one of claim 1;
an EEG amplifier configured to amplify and convert into a digital form the first voltage fluctuation, the second voltage fluctuation and the third voltage fluctuation provided by the brain-computer interface and to produce associated amplified and converted voltage fluctuations; and
a computerized device configured to determine a brain electrical activity indicator according to the associated amplified and converted voltage fluctuations.

25) The system for controlling a brain-computer interface of claim 24 wherein the computerized device is configured to produce a predetermined stimulus and the brain-computer interface is configured to measure the first voltage fluctuation, the second voltage fluctuation and the third voltage fluctuation while the predetermined stimulus is being produced.

26) The system for controlling a brain-computer interface of claim 24 wherein the predetermined stimulus is a sound stimulus.

27) The system for controlling a brain-computer interface of claim 24 wherein the system further comprises a first and second EEG brain-computer interfaces as claimed in any one of claim 1, fluctuation, the first EEG brain-computer interface being configured to provide the first voltage fluctuation, the second EEG brain-computer interface being configured to provide the second voltage fluctuation and one of the first and second EEG brain-computer interface being further configured to provide the third voltage fluctuation.

28) The system for controlling a brain-computer interface of claim 27 wherein the first EEG brain-computer interface is configured to be worn by a first ear of a user and the second EEG brain-computer interface is configured to be worn by a second ear of the user.

29) (canceled)

30) (canceled)

31) (canceled)

32) (canceled)

33) (canceled)
